(19) United States
(12) Patent Application Publication
(10) Pub. No.: US 2019/0192317 A1
(43) Pub. Date: Jun. 27, 2019

(54) PROSTHETIC LINER, PROSTHESIS SOCKET OR ORTHOSIS

(71) Applicant: OTTOBOCK SE & CO. KGAA, Duderstadt (DE)

(72) Inventors: Andre MULLER, Duderstadt (DE); Jorg KRUKENBERG, Duderstadt (DE); Sebastian BETZ, Gottingen (DE)

(73) Assignee: OTTOBOCK SE & CO. KGAA, Duderstadt (DE)

(21) Appl. No.: 16/308,971

(22) PCT Filed: Jun. 13, 2017
(86) PCT No.: PCT/EP2017/064393

§ 371 Date: Jan. 22, 2019

(30) Foreign Application Priority Data
Jun. 13, 2016 (DE) 10 2016 110 773.5

(51) Int. Cl. A61F 2/80; A61F 5/01; A61F 2/78

(52) U.S. Cl. CPC A61F 2/80; A61F 2007/0233; A61F 2/7812; A61F 5/01

(57) ABSTRACT

A prosthetic liner, prosthesis socket or orthosis with a base body, which has a volume through which gas can flow and which comprises at least one supply line and at least one evacuation line, wherein at least one compressed gas source, which is connected or can be connected to the at least one supply line such that an activation of an activation element causes compressed gas to flow out of the compressed gas source through the volume and at least partially cools the base body.

The invention relates to an orthopedic device with a base body which has a volume through which a gas can flow and which comprises at least one supply line and at least one evacuation line.

This type of orthopedic device in the form of a prosthesis socket is described, for example, in US 2015/0105865 A1. Prosthesis sockets, prosthetic liners or orthoses are referred to as orthopedic devices in the following description. One element of all these devices, hereinafter referred to as a base body, fits closely to a body part of the wearer of the orthopedic device. It is not necessary for the orthopedic device to lie directly on the body part of the wearer. It is absolutely possible that items of clothing or other orthopedic devices or objects are arranged between the respective contact surfaces of the orthopedic device and the body part of the wearer. For instance, this is the case with a prosthesis socket which features a prosthetic liner between the inner surface of the prosthesis socket and the residual limb.

Orthopedic devices often comprise a relatively large contact surface by means of which they come into contact, directly or indirectly, with the body part of the wearer. This is particularly possible with prosthesis sockets and prosthetic liners, but also with orthoses and other orthopedic devices. This extensive coverage of a body part of the wearer of the orthopedic device by the orthopedic device may lead to a stark increase in temperature at the covered points on the wearers body, which may feel unpleasant. This is often accompanied by an increase in transpiration, which is both unpleasant and unhygienic.

Therefore, in US 2015/0105865 A1 the base body is provided with a coil-shaped tube that has a supply line and an evacuation line, wherein the evacuation line is just an opening. A fan or a pump is connected to the supply line, thereby allowing air to be pumped through the channel. This enables the cooling of the base body, provided that the air is cooler than the base body, and the removal of any moisture. However, it is disadvantageous that a pump, a fan or another device that is able to move air through the channel must be provided and carried around. In addition, a power source, for example in the form of a rechargeable battery or a battery, must be transported to drive this device. This may be accompanied by potentially disruptive sounds and/or vibrations.

Furthermore, the air that is pumped through the channel must be cooler than the base body that surrounds the channel in order to produce any notable effect. On hot summer days in particular, when the air temperature can be very high, the potential cooling effect achieved by the pumped air is limited.

DE 10 2010 020 262 A1 therefore describes a device which comprises phase change materials. This refers to materials that feature a high latent heat absorption capacity, particularly during a phase change, such as the change from the liquid into the solid phase. These materials are used in the base body of a prosthesis socket, for instance, and can absorb a large amount of the heat present without experiencing an increase in temperature; this is why the heat is referred to as hidden heat or latent heat. Alongside the phase change materials, the devices described in the above-mentioned document also have a heat exchanger designed in the form of a circuit. Here, a working medium that has been cooled is guided to the phase change materials, where it absorbs part of the latent heat that is stored in the phase change materials. This results in an increase in the temperature of the working medium. The working medium is then guided to a cooling device where the heat that is has absorbed is once again removed, thereby cooling the working medium before it is fed back to the phase change materials. However, it is disadvantageous that a closed heat exchanger circuit must be available in the respective device, wherein this circuit must also comprise a separate drive unit. The potentially expensive phase change materials must also be used in such a way that they come into contact with the working medium of the heat exchanger circuit.

The invention therefore aims to improve an orthopedic device according to the generic term in claim 1 in such a way that a cooling effect can be achieved in a simple and cost-effective manner that is as independent from the external temperature as possible, without it having to be accompanied by a large, heavy, energy-intensive and possibly disruptive drive unit.

The invention solves the problem by means of an orthopedic device according to the generic term in claim 1 which is characterized by the fact that it has at least one compressed gas source that is connected or can be connected to the at least one supply line such that an activation of a supply device, by means of which a supply of compressed gas from the compressed gas source into the volume can be controlled and which in particular comprises a valve, causes compressed gas to flow out of the compressed gas source through the volume and to at least partially cool the base body.

Compressed gas should be understood to mean a gas that subjected to higher pressure than the surroundings of the orthopedic device. Here, a compressed gas source may be, for example, a canister, a can or another container in which the compressed gas is stored at a higher pressure than the surroundings of the orthopedic device. A compressed gas source may also be a container in which gas is stored in liquid form.

A supply device in this context should be understood to mean any device which has the capacity to—in particular—limit the flow of compressed gas from the compressed gas source into the volume and especially to block it completely. In the simplest configuration, this simply refers to a manually activated valve, for instance, which allows compressed gas to flow from the compressed gas source through the volume when it is open and prevents this flow when it is closed.

Upon activation of the supply device, the respective compressed gas flows from the compressed gas source through the volume. It expands while doing so and the pressure of the compressed gas reduces. This results in a decrease in temperature of the compressed gas and the base body is at least partially cooled. How much of the base body is cooled depends on the one hand on the physical properties of the material of which the base body is made. The thermal conductivity and thermal capacity of the material is thus decisive for the spread and extent of the cooling effect. On the other hand, the size of the part of the base body that is cooled also clearly depends on the course of the volume inside or on the base body.

The volume preferably has at least one tube through which substances can flow and which is arranged on or in the base body. With a prosthetic liner or prosthesis socket in particular, but also with other orthopedic devices, it is possible to arrange the tube in the material of the base body and to embed it in this material. This results in good thermal contact between the tube and the material of the base body, thereby enabling an enhancement of the cooling effect of a compressed gas flowing through the volume. In this case, it is not necessary for the tube to already be inside, i.e. as a space filled with gas, for example. The tube may also be arranged in or on the base body in a collapsed state and only caused to expand when a compressed gas flows through it.

Additionally or alternatively to a tube, the volume may also comprise a pipe or a pipeline. This may also be arranged on or in the base body.

The volume preferably runs in or on only one part of the base body. This renders it possible to restrict the cooling effect to the areas of the orthopedic device where the cooling effect is particularly desired, as these areas lie on, for example, a particularly sensitive part of the body of the wearer, or cover a body part that is especially vulnerable and sensitive to a build-up of sweat. In this way, in the case of a thigh liner or a thigh prosthetic socket for example, the base body can be equipped with the volume in the areas which run along the arteries of the wearer's residual limb. The arteries transport fresh blood coming from the wearer's torso, meaning that the blood is at the relatively high body temperature; therefore, these points (of the body) heat up and overheat more quickly than other points that are not supplied with as much arterial blood.

The at least one compressed gas source is preferably attached to the base body in such a way that it can be detached. In this case, the compressed gas source may especially preferably be a canister or a can that contains pressurized gas and, in this special configuration, can be arranged on the base body such that it can be detached. Of course, the arrangement on another component of the orthopedic device is possible, wherein the advantages of this configuration are always achieved if the compressed gas source is attached such that it be detached. The compressed gas source can then be used until it is empty or contains only a little pressurized gas. It can then be detached from the base body or another component of the orthopedic device and replaced by another full compressed gas source. This renders operation especially easy. The wearer of the orthopedic device only has to carry a sufficient supply of compressed gas sources which can be attached to the base body or another element of the orthopedic device by technically simple means in a few easy steps. In this case, following the activation of the activation element, gas—which produces the desired cooling effect—flows through the volume, without requiring a separate energy source for a pumping device or an engine-driven pump, which may cause disruptive noises.

Of course, it is not necessary to attach the compressed gas source to the pressure component. If the orthopedic device is, for instance, a prosthesis socket system with, for example, a liner and a prosthesis socket, the base body would be situated in the liner with the volume through which the gas flows. In this case, it is advantageous to arrange the compressed gas source on the prosthesis socket, for example. However, it is also possible to arrange the compressed gas source not on another component of the orthopedic device, but for instance on a belt worn by the wearer of the orthopedic device.

In a preferred configuration, the supply device comprises an activation element by means of which the supply device and especially its valve can be activated, wherein the activation element can be activated manually and is, in particular, a button. As soon as the wearer of this type of orthopedic device wants to cool down, he can activate the manual activation button and thus cause compressed gas to flow from the compressed gas source through the volume, thereby bringing about the desired cooling. The activation element, in particular the button, can cause a shutter or valve on the compressed gas source to open so that in this state, when the activation element is activated, the compressed gas can escape from the compressed gas source. If the activation element is no longer activated, i.e. for instance the button is released, the flow of gas stops because the valve or the shutter is closed.

Alternatively or additionally, the device may comprise at least one sensor for recording measured values and an electric or electronic control unit and/or regulation unit, which activates the supply device, especially the activation element, depending on the measured values. In this case, the wearer of the orthopedic device does not need to manually or deliberately activate the activation element. The sensor preferably features a temperature sensor and/or a pressure sensor, particularly an air pressure sensor and/or a moisture sensor and/or a stress sensor and/or inertial sensors, such as at least one acceleration sensor.

The temperature sensor may, for instance, measure the temperature between the orthopedic device and the body part of the wearer of the device, wherein the electric or electronic control unit activates the activation element if a preset limit is exceeded, thereby enabling compressed gas to flow from the compressed gas source into the volume; this produces the cooling effect and reduces the temperature.

A moisture sensor can be used to determine the moisture, for example the amount of sweat that has built up, between the orthopedic device and the body part of the wearer of the device. In this case, if a preset limit is exceeded, the electric or electronic control device can also activate the activation element. If the sensor comprises at least one stress sensor, it can for example measure or count the number of steps or other stressful situations experienced by a body part on which the orthopedic device is worn. As soon as a preset number of stress cycles is reached or exceeded, the electronic or electric control unit activates the activation element.

In a configuration that is structurally particularly simple, the evacuation line of the volume connects the volume with the device surroundings. The evacuation line refers, for instance, to an outward-facing opening, meaning that the compressed gas that flows through the volume can leave the volume through the evacuation line, thereby also leaving the orthopedic device.

The device preferably refers to a prosthetic liner, a prosthesis socket or an orthosis.

In a preferred configuration, the base body of the device is designed to be arranged directly on the skin of a wearer of the orthopedic device and the volume has at least one opening, which in this case is sealed by the skin of the wearer. With this type of configuration, the orthopedic device may refer to a liner, for example, which for instance comprises an airtight outer layer on its outer side and one or several openings on its inner side, wherein the inner side, in which the at least one opening is found, is situated at a distance from the airtight outer layer, for instance by way of a spacer fabric or a 3D woven fabric or textile. It is even possible to allow the inner side to only come into contact with the skin of the wearer at certain points. In this case, the volume in the base body is confined by the airtight layer on the outer side, whereas it is only confined on the inner side when the orthopedic device, i.e. the liner in the present case, is put in place and the at least one opening is sealed by the skin of the wearer of the device. The advantage of this is that the gas which is guided through the volume comes into direct contact with the skin of the wearer and its cooling effect can begin immediately.

The invention also solves the problem via a compressed gas source, especially a gas cylinder or liquid gas cylinder for an orthopedic device as described here. The term “cylinder” here does not pose any restrictions with regards to the geometric shape and/or the material of the compressed gas cylinder.

In the following, an example of an embodiment of the present invention will be explained in more detail by way of the attached drawings: They show:

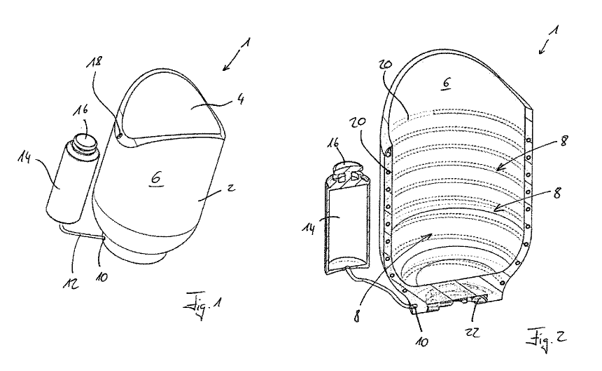

FIG. 1—a schematic depiction of an orthopedic device according to a first example of an embodiment of the present invention,

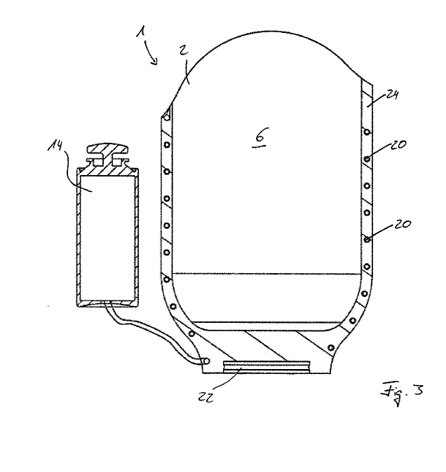

FIG. 2—a sectional view of the device from FIG. 1,

FIG. 3—another sectional view of the orthopedic device,

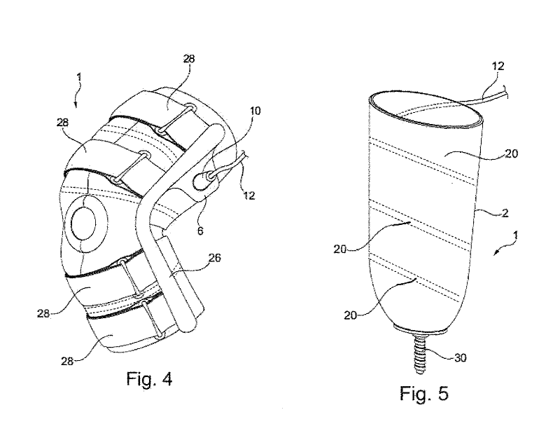

FIG. 4—a schematic depiction of an orthopedic device according to another example of an embodiment of the present invention,

FIG. 5—a schematic representation of a further orthopedic device,

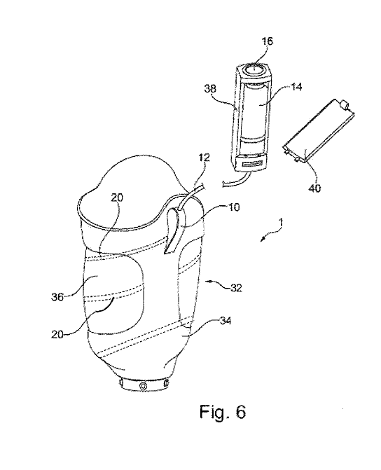

FIG. 6—a further orthopedic device according to an example of an embodiment of the present invention and

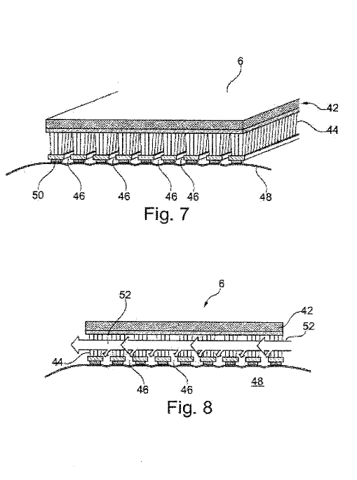

FIGS. 7 and 8—a schematic section from a further orthopedic device.

FIG. 1 depicts an orthopedic device 1 according to a first example of an embodiment of the present invention. It is a prosthetic liner 2 with a proximal opening 4 into which a residual limb can be introduced. A volume 8, which cannot be seen in FIG. 1, is located in the base body 6. However, the volume has a supply line 10 that is connected to a compressed gas source 14 via a line 12. An activation element 16 is situated in the upper part of the compressed gas source 14 depicted in FIG. 1; in the example of an embodiment shown, this activation element is activated manually. If the activation element 16 is activated, compressed gas flows from the compressed gas source 14 via the line 12 and the supply line 10 into the volume 8. On the way there and in the volume 8 itself, the gas expands and cools the volume 8 and the surrounding base body 6. The gas then leaves the volume 8 via an evacuation line 18.

FIG. 2 shows a sectional view of the orthopedic device 1 from FIG. 1. The volume 8 is shown in the base body 6, the volume being designed as a tube 20 and arranged in the base body 6 in the shape of a spiral in the example of an embodiment shown. The activation element 16 can be used to guide compressed gas from the compressed gas source 14 into the supply line 10. An adapter element 22 is located at the distal end to which additional components of a prosthesis, such as a prosthesis socket, can be arranged.

FIG. 3 depicts a sectional view through the orthopedic device 1 which has already been shown in FIGS. 1 and 2. It is clear that the tube 20, which forms the volume 8, is arranged inside a wall 24 of the base body 6 and thereby runs through the entire base boy 6 so the cooling effect produced by gas flowing out of the compressed gas source 14 cools the whole base body 6. The adapter element 22 is depicted in the distal area of the prosthetic liner 2.

FIG. 4 depicts another orthopedic device 1 according to a further example of an embodiment of the present invention. It shows a knee orthosis, the aim of which is to stabilize the wearer's knee by way of a splint 26: this is achieved by attaching it to the wearer's leg with four belts 28. The base body 6 is situated on one side, whereby it (the base body) is arranged medially, i.e. on the inner side of the leg, in this case. It is clear that the base body 6 need not necessarily be the largest element of the orthopedic device 1 or have a special supportive effect or other such function. The base body 6 is the element in which the volume 8 is found. Compressed gas can be introduced from a compressed gas source 14—not depicted—via the line 12 that is connected to the supply line 10.

FIG. 5 depicts another prosthetic liner 2 according to a further example of an embodiment of the present invention. The dashed line indicates a tube 20 which forms the volume 8 and, as shown in FIGS. 1 to 3, is arranged in the interior of the prosthetic liner 2 in the shape of a spiral or coil. However, the distance between two adjacent turns of the tube 20 in the example of an embodiment shown in FIG. 6 is considerably greater, meaning that the intended cooling effect of the gas is weaker and appears less homogeneous to the wearer of the orthopedic device 1. In this case, gas from a compressed gas source 14—not depicted—can also be introduced into the volume 8 and the tube 20 via the line 12. A locking device 30 is located at the distal end by means of which, for example, a prosthesis socket can be fixed to the prosthetic liner 2.

FIG. 6 depicts a further example of an embodiment of an orthopedic device 1. Here, it (the orthopedic device) refers to a prosthesis socket 32 with a stiff external shaft 34 and a flexibly designed inner shaft 36. The tube 20 is situated inside, wherein compressed gas from a compressed gas source 14 can be applied to the tube via the supply line 10 and the line 12. In the example of an embodiment depicted, this (the compressed gas source) is located in a housing 38. This housing 38 features a removable lid 40, which is shown in the removed state in FIG. 6. This facilitates the access to the compressed gas source 14. The activation element 16 is found in the housing 38.

FIG. 7 depicts a section 6 of an orthopedic device according to another example of an embodiment of the present invention. It has an airtight outer layer 42 and a spacer fabric 44, which features a number of openings 46. The spacer fabric 44 lies flat on the skin 48 of the user of the orthopedic device. To this end, bonding agents 44 are provided in the contact areas of the spacer fabric 44.

FIG. 8 demonstrates the situation from FIG. 7 if compressed gas from the compressed gas source—not depicted—is guided through the base body 6 along the arrows 52. Since the compressed gas cannot penetrate the airtight outer layer 42, it is guided along this layer in FIG. 8 from right to left. In the openings 46 between the individual contact areas of the spacer fabric 44, it (the spacer fabric) comes into direct contact with the skin 48 of the user of the orthopedic device, meaning it can begin cooling immediately.

### REFERENCE LIST

1 prosthetic liner, prosthesis socket or orthosis
2 prosthetic liner
4 proximal opening
6 base body
8 volume
10 supply line
12 line
14 compressed gas source
16 activation element
18 evacuation line
20 tube
22 adapter element
24 wall
26 splint
28 belt
30 locking device
32 prosthesis socket
34 external shaft
36 inner shaft
38 housing
40 lid
42 outer layer
44 spacer fabric
46 opening
48 skin
50 bonding agents
52 arrow

1. A prosthetic liner, a prosthesis socket or an orthosis, comprising:
a base body which has a volume through which gas can flow and which comprises at least one supply line and at least one evacuation line;
a supply device having a valve;
at least one compressed gas source, which is connected or can be connected to the at least one supply line such that an activation of the supply device, by means of which a supply of compressed gas from the at least one compressed gas source into the volume can be controlled;
wherein the supply device causes compressed gas to flow out of the at least one compressed gas source through the volume and to at least partially cool the base body.

2. The prosthetic liner, prosthesis socket or orthosis according to claim 1, wherein the volume comprises at least one tube through which substances can flow and which is arranged on or in the base body.

3. The prosthetic liner, prosthesis socket or orthosis according to claim 1, wherein the volume runs in or on only one part of the base body.

4. The prosthetic liner, prosthesis socket or orthosis according to claim 1, wherein the at least one compressed gas source is detachably connected or can be detachably connected to the supply line.

5. The prosthetic liner, prosthesis socket or orthosis according to claim 1, wherein the at least one compressed gas source is detachably attached to the base body.

6. The prosthetic liner, prosthesis socket or orthosis according to claim 1, wherein the supply device comprises an activation element to activate the supply device and the valve, wherein the activation element can be activated manually and is a button.

7. The prosthetic liner, prosthesis socket or orthosis according to claim 1, further comprising at least one sensor for recording measured values and an electric or electronic control unit or regulation unit, which activates the supply device depending on the measured values.

8. The prosthetic liner, prosthesis socket or orthosis according to claim 7, wherein the at least one sensor is a temperature sensor, an air pressure sensor, a moisture sensor, a stress sensor, or inertial sensors, such as at least one acceleration sensor.

9. The prosthetic liner, prosthesis socket or orthosis according to claim 1, wherein the at least one evacuation line connects the volume with surroundings of the prosthetic liner, prosthesis socket or orthosis.

10. The prosthetic liner, prosthesis socket or orthosis according to claim 1, wherein the base body is designed to be arranged directly on skin of a wearer of the prosthetic liner, prosthesis socket or orthosis and the volume has at least one opening, which is then sealed by the skin of the wearer.

11. The prosthetic liner, prosthesis socket or orthosis according to claim 1, wherein the at least one compressed gas source is a gas canister or a liquid gas canister.

12. A prosthetic liner, a prosthesis socket or an orthosis, comprising:
a base body comprising:
a volume through which gas can flow;
at least one supply line;
at least one evacuation line;
a supply device having a valve;
at least one compressed gas source configured to connect to the at least one supply line;
wherein the supply device is operable to control flow of compressed gas from the at least one compressed gas source into the volume to at least partially cool the base body.

13. The prosthetic liner, prosthesis socket or orthosis according to claim 12, wherein the volume comprises at least one tube through which substances can flow and which is arranged on or in the base body.

14. The prosthetic liner, prosthesis socket or orthosis according to claim 12, wherein the volume runs in or on only one part of the base body.

15. The prosthetic liner, prosthesis socket or orthosis according to claim 12, wherein the at least one compressed gas source is detachably connected or can be detachably connected to the supply line.

16. The prosthetic liner, prosthesis socket or orthosis according to claim 12, wherein the at least one compressed gas source is releasably attached to the base body.

17. The prosthetic liner, prosthesis socket or orthosis according to claim 12, wherein the supply device comprises an activation element to activate the supply device and the valve, wherein the activation element can be activated manually.

18. The prosthetic liner, prosthesis socket or orthosis according to claim 12, further comprising:
at least one sensor to record measured values;
an electric or electronic control unit or regulation unit, which activates the supply device depending on the measured values.

19. The prosthetic liner, prosthesis socket or orthosis according to claim 18, wherein the at least one sensor is at least one of a temperature sensor, an air pressure sensor, a moisture sensor, a stress sensor, or inertial sensors.

20. The prosthetic liner, prosthesis socket or orthosis according to claim 12, wherein the at least one evacuation line connects the volume with surroundings of the prosthetic liner, prosthesis socket or orthosis.
